(19) United States
(12) Patent Application Publication
(10) Pub. No.: US 2010/0317636 A1
(43) Pub. Date: Dec. 16, 2010

(54) REDUCING THE RISK OF CHRONIC LUNG DISEASE IN INFANTS

(75) Inventor: Tsu-Fuh Yeh, Taipei (TW)

Correspondence Address: FISH & RICHARDSON P.C. (BO), P.O. BOX 1022, MINNEAPOLIS, MN, 55440-1022, US

(73) Assignee: China Medical University, a Taiwan corporation

(21) Appl. No.: 12/861,314

(22) Filed: Aug. 23, 2010

Related U.S. Application Data
(63) Continuation of application No. 11/391,721, filed on Mar. 27, 2006, now abandoned.

(51) Int. Cl. A61K 31/56; A61P 11/00

(52) U.S. Cl. 514/171

(57) ABSTRACT

Described herein is a method for reducing the risk of chronic lung disease in an infant suffering from respiratory distress syndrome by administering to the infant a combination of a corticosteroid having a high local to systemic anti-inflammatory activity (e.g., budesonide) and a lung surfactant.

### BACKGROUND

This application is a continuation of U.S. application Ser. No. 11/391,721, filed Mar. 27, 2006.

Respiratory distress syndrome (RDS) is the most common fatal respiratory defect in infants. It is generally caused by an inadequate supply of pulmonary surfactant, a secreted wetting agent that lubricates the small airways of the lungs and prevents them from collapsing with each breath. Therapy to treat this syndrome has greatly increased postnatal survival. Nevertheless, children that survive RDS have a high risk of developing chronic lung disease (CLD), a condition characterized by a severe inflammatory response that impedes lung development and ultimately leads to impaired breathing.

The risk of CLD in infants affected by RDS can be reduced by administering a corticosteroid within a few weeks postnatally. However, the effectiveness of postnatal corticosteroid administration is offset by the fact that it may cause adverse systemic effects, e.g., hypertension, hyperglycemia, gastrointestinal complications, and neurodevelopmental disability (e.g., cerebral palsy). Locally administering a corticosteroid to the lungs results in fewer adverse effects, but poor distribution and thus reduced efficacy.

There is an ongoing need for a treatment that effectively reduces the risk of CLD in infants suffering from RDS, but does not cause adverse systemic effects.

### SUMMARY

The present invention is based, in part, on the unexpected finding that a corticosteroid having a high ratio of local to systemic anti-inflammatory activity (“HLSA corticosteroid”) and an exogenous lung surfactant can be combined and locally administered together to effectively reduce the risk of CLD in an infant suffering from RDS, without causing any adverse effects.

Accordingly, one aspect of the invention relates to a method for reducing the risk of a chronic lung disease in an infant suffering from respiratory distress syndrome. The method includes administering intratracheally to the infant an effective combination of a corticosteroid having a high ratio of local to systemic anti-inflammatory activity and a lung surfactant. The corticosteroid can be budesonide, ciclesonide, mometasone furoate, or fluticasone propionate. The effective combination can be administered to the infant at any time less than twelve hours after birth (i.e., as soon after diagnosis as possible). An effective combination of any of the above mentioned corticosteroids (e.g., budesonide) and a lung surfactant can contain a mass ratio of corticosteroid to lung surfactant between 1:1000 and 1:50 (e.g., between 1:600 to 1:250). The administration can be repeated at regular intervals until the infant has an oxygen index of less than 0.4.

Other features or advantages of the present invention will be apparent from the following detailed description, and also from the claims.

### DETAILED DESCRIPTION

A method is described herein for reducing the risk of CLD in an infant suffering from RDS. The method includes intratracheal administration of an effective combination of an HLSA corticosteroid and a lung surfactant to an infant suffering from RDS.

The term “an effective combination” refers to a mixture of an HLSA corticosteroid and a lung surfactant, the amount of each component and its proportion to other being such as to confer a prophylactic or therapeutic effect on a treated infant. Effective doses will vary, as recognized by those skilled in the art, depending on the severity of the condition to be treated, the presence of other health conditions, previous treatments, and the possibility of co-usage with other prophylactic or therapeutic treatment.

HLSA corticosteroids include, e.g., budesonide, mometasone furoate, ciclesonide, and fluticasone propionate. Budesonide is an epimeric mixture of a non-halogenated glucocorticoid, 16 alpha, 17 alpha-(22R,S)-propylmethylenedioxypregna-1,4-diene-11 beta, 21-diol-3,20-dione. Its synthesis is described in, e.g., Thalen et al. (1979), Arzneimittelforschung, 29(11):1687-90. It is rapidly degraded in the liver, consistent with its high local to systemic anti-inflammatory activity. See Ryrfeldt et al. (1982), European Journal of Respiratory Disease Supplement, 122:86-95. Budesonide is a white to off-white, tasteless, odorless powder. It is practically insoluble in water and heptane, sparingly soluble in ethanol, and freely soluble in chloroform. It is commercially available, e.g., from Spectrum Chemicals and Laboratory Products (Gardena, CA). Budesonide formulations are also available, e.g., under the trade name Pulmicort® from Astrazeneca, Inc. (Wilmington, DE).

A lung surfactant is a mixture of lipids and proteins that coats the surface of the air sacs (alveoli) and reduces surface tension in the lungs. This helps to inflate and expand the air sacs during breathing and prevents their collapse. An inadequate level of surfactant, as found in premature babies, triggers increased surface tension in the lungs that underlies RDS. Thus, exogenous lung surfactants are commonly administered intratracheally as a treatment for RDS.

Lung surfactants that can be combined with an HLSA corticosteroid as described herein include those commercially available as Survanta® (beractant) from Abbott, Ltd. (Wiesbaden, Germany), Curosurf® (poractant alpha) from Chiesi Farmaceutici (Parma, Italy), and Alveofact® from Dr. Karl Thomae, Ltd. (Biberach, Germany). The biological source and components of each of these, as stated by their manufacturers, is listed in Table1.

TABLE 1
Composition of Commercially Available Surfactant Preparations

| | % Composition (wt/wt) | | |
|---|---|---|---|
| Component | Alveofact | Curosurf | Survanta |
| Phospholipids | 88 | 99 | 84 |
| PC | 72 | 78 | 62 |
| Lyso-PC | <1 | <1 | 1 |
| PG | 8 | 3.5 | 2.5 |
| Cholesterol and | 4 | 0 | Not stated |
| neutral lipids | | | |
| Free fatty acids | 0.5 | <0.5 | 6 |
| Proteins | 1.5 (only SP-B | 1 (only SP-B | 0.5-1 (only SP-B |
| | and SP-C) | and SP-C) | and SP-C) |
| Other components | Not stated | Not stated | DPPC, neutral |
| | | | lipids are added |

Alveofact, bovine lung lavage;
Curosurf, porcine lung homogenate;
Survanta, bovine lung homogenate;
SP, surfactant protein;
PC, phosphatidylcholine;
PG, phosphatidylglycerol;
DPPC, dipalmitoylphosphatidylcholine

More recently, a surfactant containing recombinant human surfactant protein has become commercially available under the trade name Surfaxin® (lucinactant) from Discovery Labs, Inc. (Warrington, PA).

An effective combination of an HLSA corticosteroid and a lung surfactant can contain a mass ratio of corticosteroid to lung surfactant between 1: 1000 and 1: 50 inclusive (e.g., between 1:600 to 1:250 or 1:800 to 1:600). An effective amount of budesonide, for example, is 0.25 mg/kg, combined with a lung surfactant in a ratio of 1:50 or less, i.e., with 12.5 mg/kg or more of a lung surfactant (e.g., 25, 50, 100, 200, or 400 mg/kg).

Preferably, the combination of HLSA corticosteroid and lung surfactant is administered to an infant 12 hours or less after birth (e.g., less than four hours after birth).

In one example, just prior to intratracheal administration, one milliliter of a 0.25 mg/ml budesonide solution is layered on top of four milliliters of a 25 mg/ml lung surfactant solution and gently mixed (e.g., by vortexing) to form a mixture. The mixture is then administered intratracheally to the infant in four aliquots, each aliquot being administered to the infant while in a different position: lying on the right side, lying on the left side, with the chest inclined upward at an angle of 15° , and with the chest inclined downward at an angle of 15° . After administering the mixture, the infant's lungs can be discharged with the aid of a manual resuscitator (e.g., an ambu bag) to facilitate breathing and distribution of the mixture throughout the lungs. The administration can be repeated by the same procedure over regular intervals (e.g., every eight hours) after the first administration, until the infant's oxygen index is determined to be less than 0.4.

The following specific example is to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### Example 1:

A clinical study was conducted to determine if early intratracheal administration of a mixture of budesonide and a lung surfactant to infants suffering from severe RDS would reduce their risk of developing CLD.

Infants suffering from RDS and weighing less than 1500 grams were randomly assigned to either a control group (S group) administered Survanta® plus air (Survanta® 100 mg or 4 ml/kg and air 1 ml/kg), or a combination treatment group (B+S group) administered a mixture of budesonide and Survanta® (budesonide 0.25 mg or lml/kg and Survanta® 100 mg or 4 ml/kg). In both groups, a first dose was administered upon admission to the neonatal intensive care unit, and was followed by another dose every eight hours until the infant's oxygen index was determined to be less than 0.4.

Infants in the B+S group had a significantly lower incidence of CLD than infants in the S group. Moreover, among infants from both groups that developed CLD, the mortality rate was significantly lower in those from the B+S group. Infants in the B+S group also has a significantly lower oxygen index than those in the S group on days 1-3 of the study, and a significantly lower arterial blood CO₂ partial pressure on days 1 and 3.

Importantly, except for transiently higher blood pressure in the B+S group on day 5, no significant differences were found between the two groups as to serum glucose, blood osmolarity, physical growth (body weight, body length, and head circumference), incidence of patent ductus arteriosus, or incidence of intraventricular hemorrhage.

These results show that early intratracheal administration of a mixture of budesonide with a lung surfactant significantly decreased CLD mortality rate and CLD morbidity with little adverse effect.

### Other Embodiments

All of the features disclosed in this specification may be combined in any combination. Each feature disclosed in this specification may be replaced by an alternative feature serving the same, equivalent, or similar purpose. For example, the use of wholly synthetic lung surfactants in the method described herein is also contemplated. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A method for reducing the risk of chronic lung disease in an infant suffering from respiratory distress syndrome, comprising intratracheally administering to the infant an effective combination of a corticosteroid and a lung surfactant, wherein the corticosteroid has a high ratio of local to systemic anti-inflammatory activity.

2. The method of claim 1, wherein the effective combination is administered less than twelve hours after birth.

3. The method of claim 2, wherein the corticosteroid is ciclesonide, mometasone furoate, or fluticasone propionate.

4. The method of claim 2, wherein the effective combination is administered less than eight hours after birth.

5. The method of claim 4, wherein the effective combination is administered less than four hours after birth.

6. The method of claim 5, wherein the effective combination is administered less than two hours after birth.

7. The method of claim 1, wherein the effective combination contains a mass ratio of the corticosteroid to the lung surfactant of between 1:1000 to 1:50.

8. The method of claim 7, wherein the effective combination contains a mass ratio of the corticosteroid to the lung surfactant of between 1:600 to 1:250.

9. The method of claim 1, further comprising repeating the administration at regular intervals until an oxygen index of the infant is less than 0.4.

10. The method of claim 1, wherein the corticosteroid is ciclesonide.

11. The method of claim 1, wherein the corticosteroid is mometasone furoate.

12. The method of claim 1, wherein the corticosteroid is fluticasone propionate.

13. The method of claim 1, wherein the corticosteroid is budesonide.

14. The method of claim 13, wherein the effective combination is administered less than twelve hours after birth.

15. The method of claim 14, wherein the effective combination is administered less than eight hours after birth.

16. The method of claim 15, wherein the effective combination is administered less than four hours after birth.

17. The method of claim 16, wherein the effective combination is administered less than two hours after birth.

18. The method of claim 13, wherein the effective combination contains a mass ratio of the budesonide to the lung surfactant of between 1:1000 to 1:50.

19. The method of claim 18, wherein the effective combination contains a mass ratio of the budesonide to the lung surfactant of between 1:600 to 1:250.

20. The method of claim 13, further comprising repeating the administration at regular intervals until an oxygen index of the infant is less than 0.4.

21. The method of claim 13, wherein the effective combination contains a mass ratio of the budesonide to the lung surfactant of between 1:600 to 1:250 and is administered to the infant less than 12 hours after birth, and further comprising repeating the administration until an oxygen index of the infant is determined to be less than 0.4.
